(19) United States
(12) Patent Application Publication
(10) Pub. No.: US 2023/0103592 A1
(43) Pub. Date: Apr. 6, 2023

(54) SURGICAL DEVICE AND METHOD FOR REPAIRING DAMAGED FLEXOR TENDONS

(71) Applicants: Ahmadreza Afshar, Urmia (IR); Mohamad Javad Shariati, Mashad (IR)

(72) Inventors: Ahmadreza Afshar, Urmia (IR); Mohamad Javad Shariati, Mashad (IR)

(21) Appl. No.: 17/590,745

(22) Filed: Feb. 1, 2022

Related U.S. Application Data
(60) Provisional application No. 63/251,452, filed on Oct. 1, 2021.

(51) Int. Cl. A61F 2/08

(52) U.S. Cl. CPC A61F 2/0805

(57) ABSTRACT

A surgical device for performing a repair of lacerated or ruptured tendons is disclosed. The surgical device comprises a tip or first end, a base or second end opposite to the first end, and a length between the first and second end configured to pass at least in part through a tendon sheath. The surgical device increases in diameter from the first end to the second end over at least a portion of the length. The second end is shaped to accommodate retracted proximal end of the damaged tendons. The second end is inserted into a flexor fibro-osseous tunnel configured to distend and enlarge its pathway. The second end is sutured to the retracted proximal end and towed via the fibro-osseous tunnel, thereby providing a gentle, guided, and non-traumatic passage of the flexor tendon and retrieving the retracted proximal end of the damaged flexor tendon simultaneously to the repair zone.

### BACKGROUND OF THE INVENTION

A basic knowledge of the anatomy of the flexor tendons in hand, (flexor digitorum profundus (FDP) and flexor digitorum superficialis (FDS)) is essential to understand the surgical technique of tendon repair.

Flexor digitorum profundus (FDP) and flexor digitorum superficialis (FDS) connect the flexor muscles in forearm to digits' bone in hand. Each flexor tendon pass through the fibro-osseous tunnel, to better finger flexion and prevention of tendon bowstring. So, in any tendon laceration or tendon rupture in fingers the proximal part of tendon will be retracted to palm through the fibro-osseous tunnel, retrieving the proximal part of flexor tendon through fibro-osseous tunnel near to the distal part of lacerated tendon is crucial part of surgical flexor tendon repair.

Fibro-osseous tunnel of digits 2-5, contain, 5 annular pulleys (A1 to A5) pulleys, 3 cruciate pulleys (C1 to C3) collapsible and flexible allows the annular pulleys to approximate each other during digital flexion. The distance between A1 to A5 is variable in different patients but it is often about 8 centimeters in practice.

During the flexor tendon injury, digits lose the ability to bend. Particularly, flexor tendon lacerations in “No Man's Land” or Verdan Zone I and II lead to retraction of the flexor tendon toward the palm. Retrieving the flexor tendon to the surgical repair site in the finger with a minimum injury to soft tissues specially the fibro-osseous tunnel is a challenging step of the flexor tendon repair, particularly if there is associated damage to either the end of the tendon or the flexor sheath.

In surgical repair of lacerated flexor digitorum profundus (FDP) and flexor digitorum superficialis (FDS), tendon adhesion can compromise the result of surgery.

Although tendon adhesions occur and are associated with tendon injury and healing, they are not believed to be essential to the tendon repair process itself, tendon injury with injury to the synovial sheath combined with immobilization leads to extensive adhesions. annular pulley component of the fibro-osseous sheath, especially the A2 and A4 pulleys, should be preserved to prevent of adhesion.

Damages to the tendons' ends and flexor fibro-osseous tunnel can compromise the success of the flexor tendon surgery. The prognosis for a flexor tendon repair specially in the zone I and II flexor tendons is more predictable and generally better when the repair has taken place with a minimum trauma to the flexor tendon and its sheath.

Only a few attempts have been made to address the problems cited in the background. For example, Jarvis et al. (2002) discloses an inflated Fogarty catheter to dilate the tunnel and retrieve the flexor tendon, whereas others including Kilgore et al. (1971) and Wong and McGrouther (2014) recommend distension of the flexor tendon sheath with fluid.

Though there are different types of devices and techniques described in the above literature to retrieve the flexor tendon through the flexor sheath, the concept of simultaneous distention of the sheath along with retrieval of the flexor tendon is novel and unique.

Henceforth, there is a need for a surgical device and method for performing repair or of the damaged tendon in the fingers. Also, there is a need for a surgical device with several increasing sizes to let the easy passage of the edematous retracted flexor tendon. Further, there is a need for a method to facilitate the handling of a lacerated or ruptured flexor tendon during surgery and improves the outcomes of the flexor tendon repair, especially in the zone I and II flexor tendons.

### SUMMARY OF THE INVENTION

The present invention discloses relates to a device and method for facilitating the repair of damaged tendons. The present invention further discloses a surgical device and method for use in distension of the fibro-osseous tunnel and retrieval of the flexor tendon in the finger and similar surgical procedures. Further, the device and method assist in surgical repair and of damaged finger flexor tendons (FDS and FDP).

In one embodiment, the surgical device is a stepped sleeve tube or stepped sleeve rod for distension of the flexor sheath for retrieval of the flexor tendon in the finger. In one embodiment, the surgical device is a tendon passer tube with stepped increasing in tube's diameter. In one embodiment, the surgical device is designed in a shape that includes, but is not limited to, an elongated tubular configuration with a consequence increase of diameter in each step with consistent diameter between each step. In one embodiment, the surgical device is any kind of flexible tubing.

In one embodiment, the tubing can be one of a pediatric feeding tubing, an intravenous connecting tubing, a nasogastric tubing, a nelaton suction tubing, or a silicon rod. In one embodiment, the surgical device has a consistency similar to the nelaton catheter tubes. In another embodiment of the present invention, the tube or rod is flexible and made from silicon. Applicant herein also incorporates the content of Applicant's recently published article in the esteemed Journal of Hand Surgery (Afshar and Shariati, Stepped sleeve rod for flexor sheath distension and tendon retrieval following flexor tendon injury, The Journal of Hand Surgery, European volume, Dec. 21, 2021).

In one embodiment, the surgical device comprises a tip or first end, a base or second end opposite to the first end, and a length between the first end and the second end. In one embodiment, the surgical device increases in thickness from the first end to the second end over a portion of the length. In one embodiment, the surgical device has a constant cross-section and diameter throughout the length between two consequence steps. it should be emphasized that this tube is not funnel shape, whatever the tube's diameter is consistent between steps, it is increased in each step. It should be not a funnel tube, the tube equally descends the whole length of fibro-osseous tunnel, so this tube was designed with stepped incensement in diameter in certain interval with constant diameter between two consequence steps and the interval between each consecutive step would be larger equals than whole length of fibro-osseous tunnel (in this design it considered 8 cm)

In one embodiment, the surgical device has a dimension of about 48 cm length with a stepped increment of diameter configured to probe, distend, and enlarge a collapsed and contracted flexor fibro-osseous tunnel smoothly. In one embodiment, the surgical device is collapsible, pliable, smooth, malleable and flexible that ensures gentle enlargement and distension of the fibro-osseous tunnel without affecting/disrupting the fibro-osseous sheath. The tube's consistent diameter between steps, result a symmetric increment of diameter of fibro-osseous tunnel. In other hand, the distance between each step would be equal or more than A1 to A5 distance. In one embodiment, the flexible and smooth material with stepped increment of diameter of the surgical device enlarges and distends the fibro-osseous tunnel gently to the surgeon's desired level based on size of lacerated end of flexor tendon.

In one embodiment, the surgical device comprises a plurality of segments along its length. In one embodiment, the surgical device has six segments. Each segment has a dimension of about 8 cm length. In one embodiment, each segment has the increasing diameter from the tip or first end to the base or second end. Each segment is defined by one or more junction points. In one embodiment, each segment increases its diameter by 2 French (fr) continuously in every 8 cm segment with respect to the junction points until reaches 16 French (5.3 mm) at the last 8 cm segment. In one embodiment, the surgical device has a diameter of about 6 French (2 mm) at its first 8 cm segment, and its diameter increases by 2 French stepped in every 8 cm segment until reaches 16 French (5.3 mm) at the last 8 cm segment from left to right.

In one embodiment, the surgical device is used to repair the laceration or rupture of a flexor digitorum profundus (FDP) and flexor digitorum superficial (FDS) in damaged finger. In one embodiment, the FDP and FDS has a distal end and a proximal cut end or retracted proximal end. In one embodiment, a collapsed and contracted flexor tendon through fibro-osseous tunnel of the damaged finger is explored through a skin incision. In one embodiment, the retracted proximal end of flexor tendon could be inserted and sutured into the lumen of the second end of the surgical device. The sutured retracted proximal end is guided and towed through the fibro-osseous tunnel to reach to the distal end of lacerated or ruptured flexor tendon (FDS or FDP).

In one embodiment, the surgical device comprises a tip or first end, a base or second end opposite to the first end, and a length between the first end and the second end. In one embodiment, the surgical device is inserted via the collapsed and contracted fibro-osseous tunnel. In one embodiment, the second end is inserted into the fibro-osseous tunnel configured to distend and enlarge its pathway. (the fibro-osseous tunnel) In one embodiment, the surgical device is inserted via a distal or proximal window (openings) of the fibro-osseous tunnel to distend the tunnel. In one embodiment, the tube is insert ed via proximal widow to retrieve the damaged flexor tendon.

In one embodiment, the surgical device is passed from proximal window to distal window while retrieving the proximal end of lacerated flexor tendon. In one embodiment, the second end is inserted into the fibro-osseous tunnel at the distal end of the FDP or FDS. In one embodiment, the second end is inserted along its length into the fibro-osseous tunnel configured to distend and enlarge the collapsed and contracted fibro-osseous tunnel smoothly, thereby providing a non-traumatic passage of the flexor tendon through the fibro-osseous tunnel in the digit. In one embodiment, the retracted proximal end of lacerated FDP or FDS is laid about the A1 pulley in the palm.

In one embodiment, the second end of the surgical device is shaped/fashioned to a fish-mouth or shark-mouth to accommodate the distracted proximal end of the flexor tendon. In one embodiment, the fish-mouth opening at the second end is inserted and/or sutured/attached to the distracted proximal end of the flexor tendon. In one embodiment, the fish-mouth configuration of the surgical device is fashioned to prevent fraying and bunching the flexor tendon. In one embodiment, the proximal end of lacerated flexor tendon is smoothly passed through the fibro-osseous tunnel. In one embodiment, the second end is sutured intraluminally or end-to-end to the retracted proximal end of the flexor tendon according to the proportionated cross-section of the flexor tendon and second end of the surgical device. In one embodiment, the repair or of damaged tendons is performed in the zone I and II of the flexor tendon. In one embodiment, the surgical device could be used in fresh and acute, delayed, secondary tendon repairs as well as tendon reconstruction in zone I and II.

In one embodiment, a method of performing repair and of damaged flexor tendon comprises the following steps. At one step, a surgical device is provided to perform repair and of damaged flexor tendon in fingers. At another step, a collapsed and contracted fibro-osseous tunnel is explored through a skin incision. At another step, a proximal cut end or retracted proximal end of a damaged flexor digitorum profundus (FDP) and flexor tendon superficial (FDS) is positioned about a fibro-osseous tunnel another step, the surgical device is inserted via the first end along its length into the fibro-osseous tunnel configured to distend and enlarge the collapsed and contracted fibro-osseous tunnel smoothly. In one embodiment, the distension and enlarging of the fibro-osseous tunnel provide a non-traumatic passage of the flexor tendon through the fibro-osseous tunnel in the digit.

At another step, the second end of the surgical device is shaped/fashioned to accommodate the retracted proximal end of the flexor tendon. In one embodiment, the second end is shaped to a fish-mouth or shark-mouth opening to accommodate the retracted proximal end of the flexor tendon, thereby preventing any fraying and bunching in the flexor tendon. At another step, the second end is sutured to the retracted proximal end. In one embodiment, the second end is sutured to the retracted proximal end by removing the unused segment from the surgical device. At another step, the surgical device is towed to enlarge the fibro-osseous tunnel and retrieve the retracted proximal end of the damaged flexor tendon simultaneously to the repair zone. In one embodiment, the surgical device is towed from the first end. In one embodiment, the repair or of damaged tendons is performed in the zone I and II of the flexor tendons.

One aspect of the present disclosure is directed to a surgical device for performing a repair or of damaged flexor tendons (FDS and FDP). The device comprises a tip or first end, a base or second end opposite to the first end, and a length between the first and second end configured to pass at least in part through a tendon sheath and fibro-osseous tunnel, wherein the surgical device increases in diameter from the first end to the second end over at least a portion of the length, wherein the second end of the surgical device is shaped to accommodate a proximal cut end or retracted proximal end of the damaged flexor tendon, wherein the second end is inserted into a collapsed and contracted flexor fibro-osseous tunnel configured to distend and enlarge its pathway, wherein the second end is sutured to the proximal cut end of the flexor tendon and towed via the fibro-osseous tunnel, thereby providing a gentle, guided, and non-traumatic passage of the flexor tendon and retrieving the retracted proximal end of the damaged flexor tendon simultaneously to the repair zone.

In one embodiment, the device is a stepped sleeve tube having an elongated tubular shape with the continuous stepped increase of diameter. In another embodiment, the device can be any kind of flexible tubing. In another embodiment, a whole or a segment of the surgical device with an appropriate diameter is used relative to the fibro-osseous tunnel and flexor tendon (FDS and FDP). In another embodiment, the retracted proximal end of the flexor tendon is sutured to the second end of the surgical device intraluminally or end-to-end according to the proportionated cross-section of the flexor tendon and second end of the surgical device. and surgeon's desired

In one embodiment, the second end is shaped to a fish-mouth or shark-mouth to accommodate the distracted proximal end of the flexor tendon. In another embodiment, the second end is shaped to a fish-mouth or shark-mouth to prevent fraying and bunching the flexor tendon. In one embodiment, the repair or of damaged tendons is performed in the zone I and II of the flexor tendons. In another embodiment, the device has a stepped increment of the diameter to probe, distend, and enlarge the collapsed and contracted fibro-osseous tunnel smoothly, thereby providing an easy, gentle, guided, and non-traumatic passage of the flexor tendon through the fibro-osseous tunnel in the digits.

Another aspect of the present disclosure is directed to a method for performing a repair or of damaged flexor tendons (FDS or FDP), comprising (a) providing a surgical device having a tip or first end, a base or second end opposite to the first end, and a length between the first and second end configured to pass at least in part through a tendon sheath or fibro-osseous tunnel; (b) exploring a fibro-osseous tunnel through a skin incision; (c) positioning a proximal cut end or retracted proximal end of a damaged flexor digitorum profundus (FDP) or flexor digitorum superficial (FDS) in a pulley or fibro-osseous tunnel; (d) inserting the second end of the surgical device along its length into the fibro-osseous tunnel configured to distend and enlarge the collapsed and contracted fibro-osseous tunnel smoothly, thereby providing a non-traumatic passage of the proximal part of damaged flexor tendon through the fibro-osseous tunnel in the digit; (e) accommodating the retracted proximal end of the flexor tendon at the second end of the surgical device; (f) suturing the second end of the surgical device to the retracted proximal end of the flexor tendon; and (g) towing the surgical device from the first end, thereby enlarging the fibro-osseous tunnel and retrieve the retracted proximal end of the damaged flexor tendon simultaneously to the repair zone.

In one embodiment, the surgical device is a stepped sleeve tube having an elongated tubular shape with the stepped increase of diameter. In one embodiment, the surgical device increases in diameter from the first end to the second end over a portion of its length. In one embodiment, the second end of the surgical device is sutured to the retracted proximal end of the flexor tendon by removing the unused segment from the surgical device. In one embodiment, the second end is shaped to a fish-mouth or shark-mouth to accommodate the retracted proximal end of the flexor tendon, thereby preventing any fraying and bunching in the flexor tendon. In a related embodiment, the repair or of damaged tendons is performed in the zone I and II of the flexor tendons.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF DRAWINGS

FIG. 1 exemplarily illustrates a perspective view of a surgical device, according to an embodiment of the present invention;

FIG. 2 exemplarily illustrates a perspective view of laceration of finger flexor tendon and a fibro-osseous sheath, according to one embodiment of the present invention;

FIG. 3 exemplarily illustrates removing of an unused segment of the surgical device, according to one embodiment of the present invention;

FIG. 4 exemplarily illustrates suturing of a damaged flexor tendon to one end of the surgical device, according to one embodiment of the present invention;

FIG. 5 exemplarily illustrates retrieving of the proximal part of lacerated flexor tendon via the fibro-osseous tunnel, according to one embodiment of the present invention;

FIG. 6 exemplarily illustrates repairing of the flexor tendon, according to one embodiment of the present invention; and

FIG. 7 exemplarily illustrates a method for performing a repair and of damaged flexor tendons, according to one embodiment of the present invention.

### DETAILED DESCRIPTION

The present invention generally relates to a device and method for facilitating repair of a lacerated or ruptured finger's flexor tendon (FDS and FDP) and more particularly relates to a surgical device and method for use in distension of the fibro-osseous tunnel and retrieval of the flexor tendon in the finger and similar surgical procedures. This device and method assist in surgical repair and of damaged tendons.

A description of embodiments of the present invention will now be given with reference to the figures. It is expected that the present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes that come within the meaning and range of equivalency of the claims are to be embraced within their scope.

Before any embodiments of the invention are explained in detail, it is to be understood that the invention is not limited in its application to the details of construction nor to the arrangement of components set forth in the following description or illustrated in the drawings. The invention is capable of other embodiments and of being practiced or of being carried out in various ways.

The present invention discloses a surgical device for performing a repair or of lacerated or ruptured flexor tendons in the zone I and II flexor tendons. The present invention further discloses a stepped sleeve tube for distension of a fibro-osseous tunnel and retrieval of the flexor tendon in the finger.

Applicant incorporates herein the content of Applicant's recently published article in the esteemed Journal of Hand Surgery (Afshar and Shariyati, Stepped sleeve rod for flexor sheath distension and tendon retrieval following flexor tendon injury, The Journal of Hand Surgery, European volume, Dec. 21, 2021).

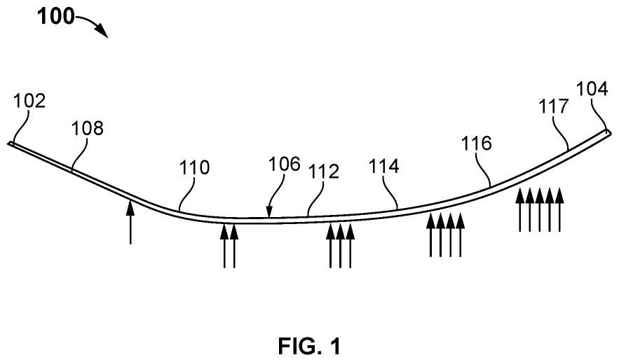

Referring to FIG. 1, a perspective view of a surgical device 100 according to an embodiment of the present invention is disclosed. In one embodiment, the surgical device 100 is a stepped sleeve tube or stepped sleeve rod for distension of the flexor sheath for retrieval of the flexor tendon in the finger. In one embodiment, the surgical device 100 is a new stepped sleeve tendon passer tube. In one embodiment, the surgical device 100 is designed in a shape that includes, but is not limited to, an elongated tubular configuration with a continuous increase of diameter. In one embodiment, the surgical device 100 is any one of flexible tubing.

In one embodiment, the surgical device 100 comprises a tip or first end 102, a base or second end 104 opposite to the first end, and a length 106 between the first end 102 and the second end 104. In one embodiment, the surgical device 100 increases in thickness from the first end 102 to the second end 104 over a portion of the length 106. In one embodiment, the surgical device 100 has a constant cross-section and diameter throughout the length 106.

In one embodiment, the surgical device 100 has a dimension of about 48 cm length with a stepped increment of diameter configured to probe, distend, and enlarge a collapsed and contracted flexor fibro-osseous tunnel 122 smoothly. In one embodiment, the stepped increment of diameter of the surgical device 100 enlarges and distends the fibro-osseous tunnel 122 gently to the surgeon's desired level. In one embodiment, the surgical device 100 is collapsible, pliable, and malleable that ensures gentle enlargement and distension of the fibro-osseous tunnel 122 without affecting/disrupting the fibro-osseous sheath. In one embodiment, the surgical device 100 comprises a plurality of segments along its length 106.

The surgical device 100 has an increasing diameter for each segment from the first end 102 at the left end to the second end 104 at the right end. Each segment is defined by one or more junction points (⬆, ⬆⬆, ⬆⬆⬆, ⬆⬆⬆⬆, and ⬆⬆⬆⬆⬆). From left to right: ⬆ junction of 6 and 8 Frenches; ⬆⬆ junction of 8 and 10 Frenches; ⬆⬆⬆ junction of 10 and 12 Frenches; ⬆⬆⬆⬆ junction of 12 and 14 Frenches, and ⬆⬆⬆⬆⬆ junction of 14 and 16 Frenches. In one embodiment, each segment increases its diameter by 2 French (fr) continuously in every 8 cm segment with respect to the junction points (⬆, ⬆⬆, ⬆⬆⬆, ⬆⬆⬆⬆, and ⬆⬆⬆⬆⬆) until reaches 16 French (5.3 mm) at the last 8 cm segment. In one embodiment, the surgical device 100 has a diameter of about 6 French (2 mm) at its first 8 cm segment, and its diameter increases by 2 French continuously in every 8 cm segment until reaches 16 French (5.3 mm) at the last 8 cm segment from left to right.

In one embodiment, the surgical device 100 has six segments. In one embodiment, the surgical device 100 comprises a first segment 108, a second segment 110, a third segment 112, a fourth segment 114, a fifth segment 116, and a sixth segment 117. Each segment (108, 110, 112, 114, 116, and 117) has a dimension of about 8 cm length. In one embodiment, each segment has the increasing diameter from the tip or first end 102 to the base or second end 104.

The first segment 108 has an increasing diameter with respect to the tip or first end 102 and the first junction (⬆) of ch/fr 6 and 8. The second segment 110 has an increasing diameter with respect to the first junction (⬆) and the second junction (⬆⬆) of ch/fr 8 and 10. The third segment 112 has an increasing diameter with respect to the second junction (⬆⬆) and the third junction (⬆⬆⬆) of ch/fr 10 and 12. The fourth segment 114 has an increasing diameter with respect to the third junction (⬆⬆⬆) and the fourth junction (⬆⬆⬆⬆) of ch/fr 12 and 14. The fifth segment 116 has an increasing diameter with respect to the fourth junction (⬆⬆⬆⬆) and the fifth junction (⬆⬆⬆⬆⬆) of ch/fr 14 and 16. The sixth segment 117 has an increasing diameter with respect to the fifth junction (⬆⬆⬆⬆⬆) and the base or second end 104. Further, each segment has different color that is adopted from the standard of medical standard French sizes colorings.

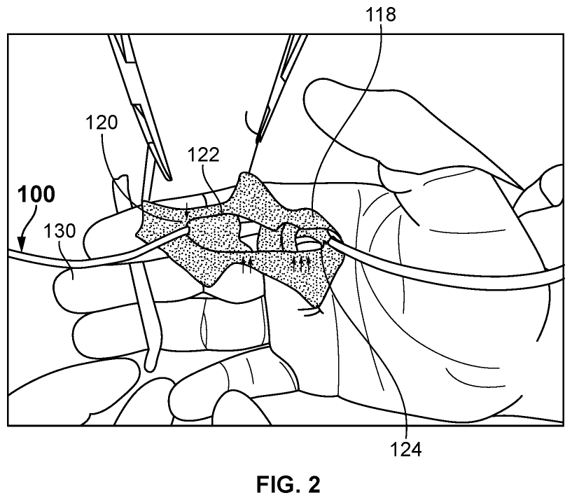

Referring to FIG. 2, a perspective view of laceration of a flexor digitorum profundus (FDP) or sperficialis (FDS) 118 in a damaged finger 130, according to one embodiment of the present invention. 118. The surgical device 100 is used in a secondary flexor digitorum profundus repair. In one embodiment, the FDP or FDS 118 has a distal end 120 and a proximal cut end or refracted proximal end 124. In on embodiment, a collapsed and contracted flexor fibro-osseous tunnel or fibro-osseous tunnel 122 of the damaged finger 130 is explored through a Bruner skin incision. The fibro-osseous tunnel is enlarged to about 14 French and the remaining unused 16 French segment of the surgical device 100 is removed. In one embodiment, the retracted proximal end 124 could be inserted and sutured into the lumen of the second end 104 of the surgical device 100. The sutured retracted proximal end 124 is guided and towed through the fibro-osseous tunnel 122.

In one embodiment, the surgical device 100 comprises a tip or first end 102, a base or second end 104 opposite to the first end, and a length 106 between the first end 102 and the second end 104. In one embodiment, the surgical device 100 is inserted via the collapsed and contracted fibro-osseous tunnel 122. In one embodiment, the second end 104 of the surgical device 100 is inserted into the fibro-osseous tunnel 122 configured to distend and enlarge its pathway. In one embodiment, the surgical device 100 is inserted via a distal or proximal window (opening) of the fibro-osseous tunnel 122 to retrieve the damaged flexor tendon. to distal part of lacerated or ruptured flexor tendon.

In one embodiment, the surgical device 100 is malleable that may be introduced from a distal or proximal window of the flexor sheath to enable probing and gentle smooth enlargement of the collapsed and contracted flexor sheath, without disrupting the sheath. Either the whole length of the surgical device 100 or an appropriate segment with the diameter corresponding to the sheath/tendon cross section may be used at the discretion of the operating surgeon. In one embodiment, the base end or second end 104 of the surgical device 100 may be shaped to a fish-mouth to accommodate the retracted proximal end of the flexor tendon, thereby preventing any fraying, or bunching up of the tendon as it is pulled through the sheath.

For example, the surgical device 100 is passed from a proximal window to distal window while retrieving the flexor tendon. In one embodiment, the second end 104 is inserted into the fibro-osseous tunnel 122 at the distal end 120 of the FDP or FDS 118. In one embodiment, the second end 104 is inserted along its length into the fibro-osseous tunnel 122 configured to distend and enlarge the collapsed and contracted fibro-osseous tunnel 122 smoothly, thereby providing a non-traumatic passage of the flexor tendon through the fibro-osseous tunnel 122 in the digit. In one embodiment, the retracted proximal end 124 of retracted FDP or FDS 118 is laid about the A1 pulley of the finger.

One embodiment of the surgical device is a stepped sleeve tube having an elongated tubular shape with the continuous stepped increase of diameter. In another embodiment, the surgical device has one or more segments with the continuous stepped increase of diameter.

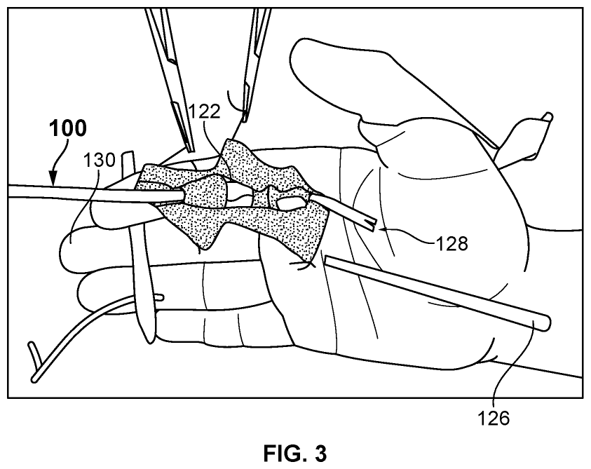

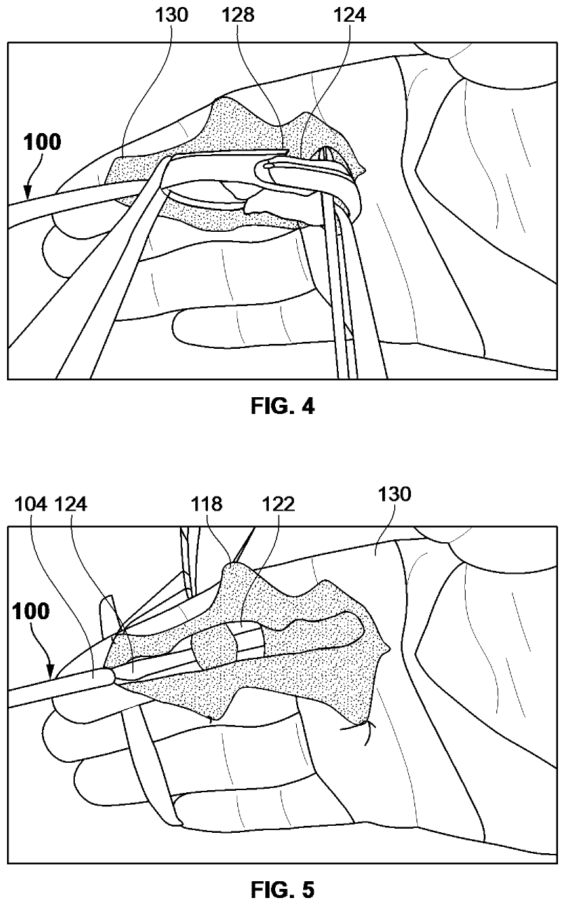

Referring to FIGS. 3-4, attaching the retracted proximal end 124 of the FDP or FDS 118 to the surgical device 100, according to one embodiment of the present invention. The surgical device 100 is inserted into the fibro-osseous tunnel 122 via its second end 104. In one embodiment, the width of the fibro-osseous tunnel 122 is enlarged adequately with the 14 fr segment and 16 fr segment is not used. The unused part 126 is removed then from the surgical device 100. In one embodiment, the segments as a whole or in part of the surgical device 100 with an appropriate diameter relative to the flexor fibro-osseous tunnel and the diameter of flexor tendon (FDS or FDP) 122 are used and passed through the fibro-osseous tunnel 122 upon the surgeon's discern and judgment.

In one embodiment, the second end 104 of the surgical device 100 is shaped/fashioned to a fish-mouth or shark-mouth opening 128 to accommodate the distracted proximal end 124 of the flexor tendon. In one embodiment, the fish-mouth opening 128 at the second end 104 is inserted and/or sutured/attached to the distracted proximal end 124 of the flexor tendon. In one embodiment, the fish-mouth configuration 128 of the surgical device 100 is fashioned to prevent fraying and bunching the flexor tendon according to the surgeon's desire.

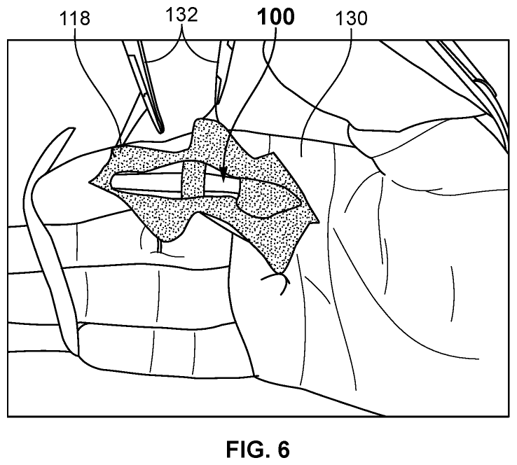

Referring to FIGS. 5-6, retrieval and repair of the flexor tendon, according to one embodiment of the present invention. In one embodiment, the second end 104 of the surgical device 100 is inserted into the collapsed and contracted flexor fibro-osseous tunnel 122 configured to distend and enlarge its pathway. In one embodiment, the surgical device 100 has a stepped continuous increment of the diameter to probe, distend, and enlarge the collapsed and contracted fibro-osseous tunnel 122 smoothly, thereby providing an easy, gentle, guided, and non-traumatic passage of the flexor tendon through the fibro-osseous tunnel 122 in the digits.

The second end 104 is, in one example, sutured to the retracted proximal end 124 of the flexor tendon and towed via the fibro-osseous tunnel 122, thereby providing a gentle, guided, and non-traumatic passage of the flexor tendon and retrieving the retracted proximal end of the damaged flexor tendon simultaneously to the repair zone. In one embodiment, the retracted proximal end 124 could be inserted and sutured into the lumen of the second end 104 of the surgical device 100.

The sutured retracted proximal end 124 is guided and towed through the fibro-osseous tunnel 122. In one embodiment, the second end 104 is sutured intraluminally or end-to-end to the retracted proximal end 124 of the flexor tendon according to the proportionated cross-section of the flexor tendon and second end 104 of the surgical device 100. In one embodiment, the second end 104 is sutured to the retracted proximal end 124 using a needle 132. In one embodiment, the repair or of damaged tendons is performed in the zone I and II of the flexor tendons. In one embodiment, the surgical device 100 could be used in fresh and acute, delayed, secondary tendon repairs as well as tendon reconstruction in zone I and II.

In the acute and fresh flexor tendon laceration, the retracted end of the flexor tendon can be sutured to a plastic or silicon tubing and guided through the fibro-osseous tunnel or flexor sheath to the repair zone, which is a useful technique for retrieval. Various devices, such as a paediatric feeding tube, intravenous connecting tube, nasogastric tube, ‘Nelaton’ suction catheter or a silicon rod, can also be used for this purpose. These available devices have a constant diameter throughout their entire length.

In some stubborn acute cases, the tendon sheath (window) opening needs to be enlarged slightly with a kind of dilator, for example, a paediatric urethral dilator, to let an easy passage of the flexor tendon. In delayed (before 10 days) or secondary (before 4 weeks) flexor tendon repair tendon edema, flexor tendon sheath collapse, and tendon sheath contracture may prevent an easy and non-traumatic passage of the retracted proximal end of the flexor tendon through the fibro-osseous tunnel, particularly through a relatively long segments of the tunnel/sheath. Therefore, the fibro-osseous tunnel may need some probing, widening, dilatation, and distension by several increasing sizes of tubing, rod, or dilator sequentially to allow for the easy passage of the retracted flexor tendon.

In one embodiment of the method, the surgical device is a stepped sleeve tube having an elongated tubular shape with the stepped increase of diameter. In another embodiment of the method, the surgical device increases in diameter from the first end to the second end over a portion of its length. In another embodiment of the method, the second end of the surgical device is sutured to the retracted proximal end of the flexor tendon by removing the unused segment from the surgical device. In another embodiment, the second end is shaped to a fish-mouth or shark-mouth to accommodate the retracted proximal end of the flexor tendon, thereby preventing any fraying and bunching in the flexor tendon. In a further embodiment, the repair or of damaged tendons is performed in the zone I and II of the flexor tendons.

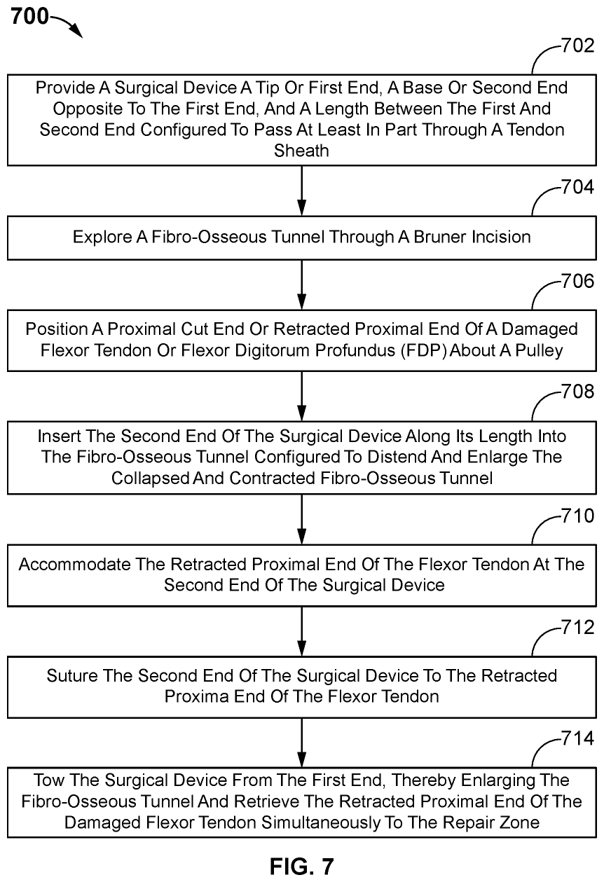

Referring to FIG. 7, a method 700 of performing repair and rehabilitation of damaged flexor tendon, according to one embodiment of the present invention. The method 700 comprises the following steps. At step 702, a surgical device 100 is provided to perform repair and of damaged flexor tendon in fingers. In one embodiment, the surgical device 100 is a stepped sleeve tube having an elongated tubular shape with the stepped increase of diameter. In one embodiment, the surgical device 100 comprises a tip or first end 102, a base or second end 104 opposite to the first end 102, and a length 106 between the first and second end (102 and 104) configured to pass at least in part through a tendon sheath or fibro-osseous tunnel 122. In one embodiment, the surgical device 100 increases in diameter from the first end 102 to the second end 104 over at least a portion of the length.

At step 704, a collapsed and contracted fibro-osseous tunnel is explored through a skin incision. At step 706, a proximal cut end or retracted proximal end 124 of a damaged flexor tendon superficialis (FDS) or flexor digitorum profundus (FDP) is positioned about a pulley. The pulley is A1 pulley. At step 708, the surgical device 100 is inserted via the second end 104 along its length into the fibro-osseous tunnel 122 configured to distend and enlarge the collapsed and contracted fibro-osseous tunnel 122 smoothly. In one embodiment, the distension and enlarging of the fibro-osseous tunnel 122 provide a non-traumatic passage of the flexor tendon through the fibro-osseous tunnel in the digit.

At step 710, the second end 104 of the surgical device 100 is shaped/fashioned to accommodate the retracted proximal end 124 of the flexor tendon. In one embodiment, the second end 104 is shaped to a fish-mouth or shark-mouth 128 to accommodate the retracted proximal end 124 of the flexor tendon, thereby preventing any fraying and bunching in the flexor tendon. At step 712, the second end 104 is sutured to the retracted proximal end 124. In one embodiment, the second end 104 is sutured to the retracted proximal end 124 by removing the unused segment 126 from the surgical device 100.

At step 714, the surgical device 100 is towed to enlarge the fibro-osseous tunnel and retrieve the retracted proximal end of the damaged flexor tendon simultaneously to the repair zone. In one embodiment, the surgical device 100 is towed from the first end 102. In one embodiment, the repair or rehabilitation of damaged tendons is performed in the zone I and II of the flexor tendons. In one embodiment, the surgical device 100 could be used in fresh and acute, delayed, secondary tendon repairs as well as tendon reconstruction in zone I and II.

Advantageously, the surgical device of the present invention is a new tendon passer tube made of inexpensive materials. The surgical device has the graded stepped diameter that combines distention and dilatation of the flexor sheath concurrent to tendon retrieval. The design of the surgical device allows it to be introduced into the flexor sheath through an entrance and an exit window, thus avoiding the need for several separate openings. This allows the flexor sheath to be dilated as a whole, rather than needing to enlarge each individual pulley separately with multiple attempts. The surgical device is used to expedites the operational procedures, save time, and costs instead of using several tubes with different sizes.

The surgical device facilitates the handling of a lacerated flexor tendon during surgery and improves the outcomes of the flexor tendon repair in the zone I and II flexor tendons by minimizing soft tissue injury specially fibro-osseous tunnel and tendon sheath. The prognosis for repairing the flexor tendon in the zone I and II is more predictable and generally better when the repair has taken place with minimum trauma to the flexor tendon and its sheath. Further, the surgical device enables better handling of a lacerated flexor tendon during surgery and, we believe, improves the outcomes of the flexor tendon repair in Zone I and II.

The insertion and towing of the surgical device into the collapsed and contracted fibro-osseous tunnel distends and dilates the fibro-osseous tunnel simultaneously with retrieving the flexor tendon. Further, the design and development of the surgical device distend, enlarge, and dilate the collapsed and contracted fibro-osseous tunnel smoothly on towing the inserted surgical device on the tunnel, thereby providing easy, gentle, and non-traumatic passage of the flexor tendon through the fibro-osseous tunnel.

The foregoing description comprise illustrative embodiments of the present invention. Having thus described exemplary embodiments of the present invention, it should be noted by those skilled in the art that the within disclosures are exemplary only, and that various other alternatives, adaptations, and modifications may be made within the scope of the present invention. Merely listing or numbering the steps of a method in a certain order does not constitute any limitation on the order of the steps of that method.

Many modifications and other embodiments of the invention will come to mind to one skilled in the art to which this invention pertains having the benefit of the teachings presented in the foregoing descriptions. Although specific terms may be employed herein, they are used only in generic and descriptive sense and not for purposes of limitation. Accordingly, the present invention is not limited to the specific embodiments illustrated herein. While the above is a complete description of the preferred embodiments of the invention, various alternatives, modifications, and equivalents may be used. Therefore, the above description and the examples should not be taken as limiting the scope of the invention, which is defined by the appended claims.

1. A surgical device for performing a repair or of damaged tendons, comprising:
a tip or first end, a base or second end opposite to the first end, and a length between the first and second end configured to pass at least in part through a tendon sheath and fibro-osseous tunnel, wherein the surgical device increases in diameter from the first end to the second end over at least a portion of the length,
wherein the second end of the surgical device is shaped to accommodate a proximal cut end or retracted proximal end of the damaged flexor tendon,
wherein the second end is inserted into a collapsed and contracted flexor fibro-osseous tunnel configured to distend and enlarge its pathway,
wherein the second end is sutured to the proximal cut end of the flexor tendon and towed via the fibro-osseous tunnel, thereby providing a gentle, guided, and non-traumatic passage of the flexor tendon and retrieving the retracted proximal end of the damaged flexor tendon simultaneously to the repair zone.

2. The surgical device of claim 1, is a stepped sleeve tube having an elongated tubular shape with the continuous stepped increase of diameter.

3. The surgical device of claim 1, has one or more segments with the continuous stepped increase of diameter.

4. The surgical device of claim 1, is any one of flexible tube (material).

5. The surgical device of claim 1, a whole or a segment of the surgical device with an appropriate diameter is used relative to the fibro-osseous tunnel and flexor tendon (FDS and FDP)

6. The surgical device of claim 1, wherein the retracted proximal end of the flexor tendon is sutured to the second end of the surgical device intraluminally or end-to-end according to the proportionated cross-section of the flexor tendon and second end of the surgical device.

7. The surgical device of claim 1, wherein the second end is shaped to a fish-mouth or shark-mouth to accommodate the distracted proximal end of the flexor tendon.

8. The surgical device of claim 1, wherein the second end is shaped to a fish-mouth or shark-mouth to prevent fraying and bunching the flexor tendon.

9. The surgical device of claim 1, wherein the repair or of damaged tendons is performed in the zone I and II of the flexor tendons.

10. The surgical device of claim 1, has a stepped increment of the diameter to probe, distend, and enlarge the collapsed and contracted fibro-osseous tunnel smoothly, thereby providing an easy, gentle, guided, and non-traumatic passage of the flexor tendon through the fibro-osseous tunnel in the digits.

11. A method for performing a repair or of damaged tendons, comprising:
providing a surgical device having a tip or first end, a base or second end opposite to the first end, and a length between the first and second end configured to pass at least in part through a tendon sheath;
exploring a fibro-osseous tunnel through a skin incision; positioning a proximal cut end or retracted proximal end of a damaged flexor tendon or flexor digitorum profundus (FDP) or flexor digitorum superficialis (FDS) about a pulley; inserting the second end of the surgical device along its length into the fibro-osseous tunnel configured to distend and enlarge the collapsed and contracted fibro-osseous tunnel smoothly, thereby providing a non-traumatic passage of the flexor tendon through the fibro-osseous tunnel in the digit;
accommodating the retracted proximal end of the flexor tendon at the second end of the surgical device;
suturing the second end of the surgical device to the retracted proximal end of the flexor tendon, and
towing the surgical device from the first end, thereby enlarging the fibro-osseous tunnel and retrieve the retracted proximal end of the damaged flexor tendon simultaneously to the repair zone.

12. The method of claim 11, wherein the surgical device is a stepped sleeve tube having an elongated tubular shape with the stepped increase of diameter.

13. The method of claim 11, wherein the surgical device increases in diameter from the first end to the second end over a portion of its length.

14. The method of claim 11, wherein the second end of the surgical device is sutured to the retracted proximal end of the flexor tendon by removing the unused segment from the surgical device.

15. The method of claim 11, wherein the second end is shaped to a fish-mouth or shark-mouth to accommodate the retracted proximal end of the flexor tendon, thereby preventing any fraying and bunching in the flexor tendon.

16. The method of claim 11, wherein the repair or of damaged tendons is performed in the zone I and II of the flexor tendons.
